(19) United States
(12) Patent Application Publication
(10) Pub. No.: US 2012/0192862 A1
(43) Pub. Date: Aug. 2, 2012

(54) PATIENT-CONTROLLED AEROSOL ADMINISTRATION

(75) Inventors: Stephen Lewis, Atlanta, GA (US); Cheryl Lewis, Atlanta, GA (US); Thomas Westfall, Riverside, CA (US); Terry Blansfield, Orange, CA (US)

(73) Assignee: CareFusion 303, Inc., San Diego, CA (US)

(21) Appl. No.: 13/018,258

(22) Filed: Jan. 31, 2011

(51) Int. Cl. A61M 11/00

(52) U.S. Cl. 128/200.23

(57) ABSTRACT

A system and method of controlling the administration of a medical substance is disclosed. An aerosol generator is configured to aerosolize a medical substance and administer the aerosolized medical substance to a patient using a ventilator. The patient is provided with a patient control interface through which the patient initiates the administration of a dose of the aerosolized medical substance. A processor is configured to control the ventilator and the aerosol generator in response to the patient control interface such that the patient controls the administration of the aerosolized medical substance in accordance with limits on the administration of the medical substance.

### BACKGROUND

1. Field

The present disclosure generally relates to systems and methods for delivery of therapeutic aerosols to a patient and, in particular, relates to control of the administration of an aerosolized medical substance by a patient using a ventilator.

2. Description of the Related Art

People who have been seriously injured or undergone major surgery may have difficulty in breathing on their own. In order to ensure that sufficient oxygen is available in the lungs for absorption, a ventilator may be used to mechanically assist or replace spontaneous breathing. Positive-pressure ventilators work by increasing the patient's airway pressure through a patient device such as a mask or an endotracheal or tracheotomy tube. The positive pressure forces air to flow into the lungs. When the ventilator reduces the pressure, the elastic contraction of the chest wall collapses the lungs and pushes a volume of air out.

Patients using a ventilator often have medications such as a bronchodilator administered to them by the nurse or other caregiver using a nebulizer or other aerosol generator connected to the inspiratory tubing. Some of these medications are therapeutic and must be administered on a fixed schedule. Other medications are provided for the comfort of the patient and are administered only at the request of the patient. These optional medications are referred to as “PRN” which is a shortened form of the Latin phrase pro re nata that translates roughly to “as the thing is needed.” It may be difficult for the patient to make this request, however, when a breathing mask or endotracheal tube is in place, and there may be a further delay between when the patient succeeds in communicating the request and when the nurse is able to gather the necessary equipment and supplies and administer the medication. It would be beneficial to the patient to be able to self-administer PRN aerosol medications in a safe and controlled manner.

### SUMMARY

The disclosed system and method describe a ventilator system that is configured to allow the patient to initiate the administration of aerosolized medications, such as a bronchodilator comprising ipratropium bromide, or medical substances, such as a wetting agent comprising 0.9% NaCl in water. In certain embodiments, the physician may prescribe one or medications that can be administered to the patient at the patient's request. The ventilator may be configured to include a minimum time interval between sequential administrations of one or more of the prescribed medications.

In certain embodiments, a method of controlling the administration of a medical substance is disclosed. The method comprises the steps of configuring an aerosol generator to aerosolize a medical substance and administer the aerosolized medical substance to a patient using a ventilator, providing the patient with a patient control interface through which a patient initiates the administration of a dose of the aerosolized medical substance, and configuring a processor to control the ventilator and the aerosol generator in response to the patient control interface such that the patient controls the administration of the aerosolized medical substance in accordance with limits on the administration of the medical substance.

In certain embodiments, a ventilation system for use by a patient is disclosed. The ventilator system comprises a patient device attached to the patient wherein the patient device is configured to introduce gas into the lungs of the patient, a gas control module fluidically coupled to the patient device wherein the gas control module is configured to controllably provide a gas to the patient device according to at least one operating parameter, an aerosol generator coupled to the patient device wherein the aerosol generator is configured to administer a dose of an aerosolized medical substance, a patient control interface configured to control the at least one operating parameter of the gas control module and to be accessible by the patient, and a processor coupled to the gas control module, the aerosol generator, and the patient control interface wherein the processor is configured to operate the gas control module and the aerosol generator in response to the patient control interface.

In certain embodiments, a computer-readable medium having computer-executable instructions stored thereon for execution by a processor to perform a method of controlling the administration of a medical substance is disclosed. The method comprises the steps of configuring an aerosol generator to aerosolize a medical substance and administer the aerosolized medical substance to a patient using a ventilator, providing the patient with a patient control interface through which a patient initiates the administration of a dose of the aerosolized medical substance, and configuring a processor to control the ventilator and the aerosol generator in response to the patient control interface such that the patient controls the administration of the aerosolized medical substance in accordance with limits on the administration of the medical substance.

In certain embodiments, an aerosol generation system for use by a patient is disclosed that comprises a patient device attached to the patient wherein the patient device is configured to introduce gas into the lungs of the patient, an aerosol generator coupled to the patient device wherein the aerosol generator is configured to administer a dose of an aerosolized medical substance through the patient device, and a patient control interface coupled to the aerosol generator and configured to be accessible by the patient wherein the patient control interface is configured to cause the aerosol generator to initiate administration of the dose of aerosolized medical substance.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 depicts a patient using a positive pressure mechanical ventilator that can be used for the system of the present disclosure.

FIG. 2 depicts an example patient control interface according to certain aspects of the present disclosure.

FIG. 3 is a block diagram of a ventilator and an aerosol generator configured to be controlled by a patient according to certain aspects of the present disclosure.

FIG. 4 is a block diagram of a ventilator controller configured to control a ventilator and an aerosol generator according to certain aspects of the present disclosure.

FIG. 5 is a flow chart of the methodology of a patient controlling the administration of a medical substance according to certain aspects of the present disclosure.

### DETAILED DESCRIPTION

It is advantageous to administer certain medical substances as an aerosol when a patient is using a ventilator. These medical substances may be medications, anesthetics, or other substances or mixtures that have a beneficial effect, such as a saline solution. As the medical substance is delivered directly to the lungs, it is particularly suited to treat lung injuries as well as alleviating discomfort due to the operation of the ventilator. Certain medical substances are prescribed as PRN which allows the patient to receive the medical substance upon request, subject to restrictions imposed by the doctor on the frequency of administration. The disclosed system enables the patient to initiate administration of a dose of a PRN medical substance within the imposed restrictions, increasing the patient's comfort while reducing the workload of the nurse.

In the following detailed description, numerous specific details are set forth to provide a full understanding of the present disclosure. It will be apparent, however, to one ordinarily skilled in the art that embodiments of the present disclosure may be practiced without some of the specific details. In other instances, well-known structures and techniques have not been shown in detail so as not to obscure the disclosure.

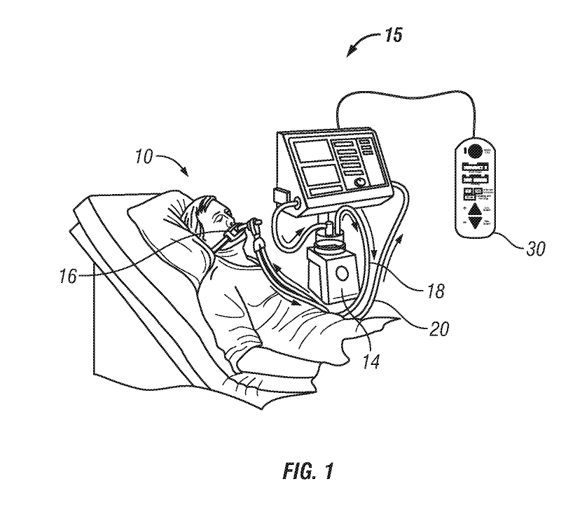

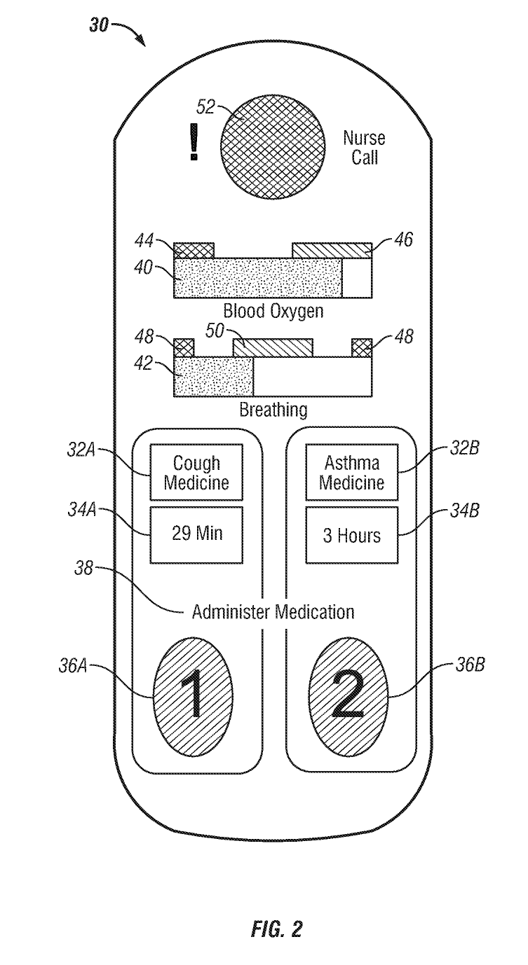

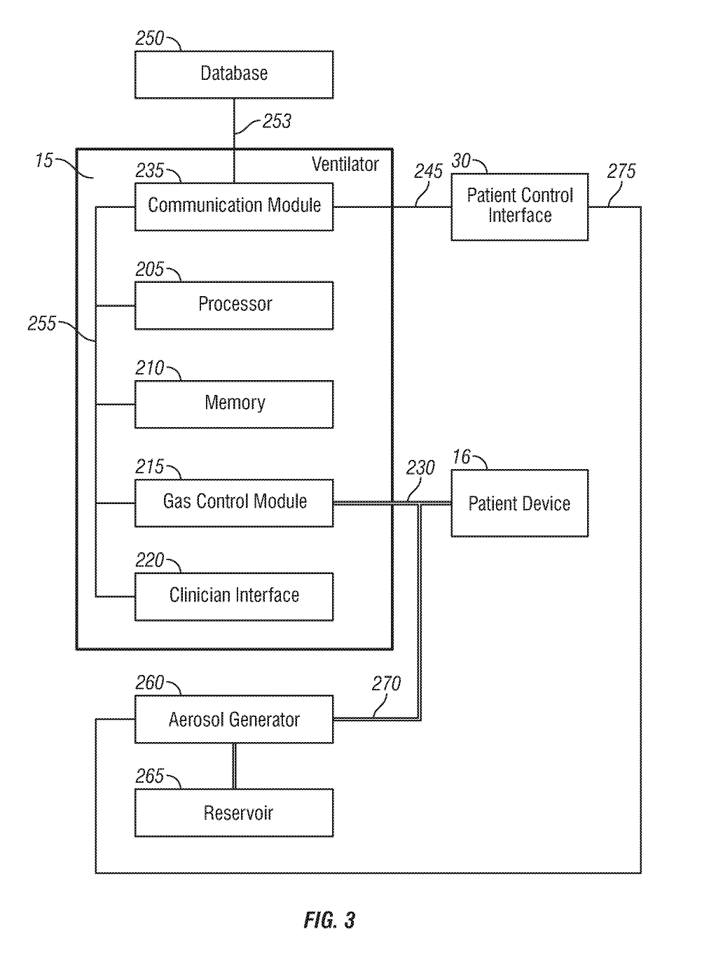

FIG. 1 depicts a patient 10 using a positive pressure mechanical ventilator 15 that can be used for the system of the present disclosure. The patient 10 is wearing a patient device 16 such as an oral endotracheal tube that is attached with straps. In other situations, alternate patient devices 16 such as a full-face or nose-and-mouth mask, a laryngeal mask, a nasal endotracheal tube, or a tracheotomy tube may be used. The ventilator 15 is, in this example, attached to the patient device 16 by an inspiratory hose 18 and an expiratory hose 20. Air from the ventilator 15 passes through, in this example, a conditioning unit 14 before entering inspiratory hose 18 so that the air that is supplied to the patient 10 is at a specified temperature and humidity. In certain embodiments, an aerosol generator (not shown) is connected at or near the patient device 16. In certain other embodiments, the aerosol generator is connected to or just after the conditioning unit 14. In certain other embodiments, the aerosol generator is connected to the inspiratory hose 18 via a “Y” connector. This is discussed below in more detail in FIG. 3. The ventilator 15 also includes a patient control interface 30 that enables the patient to self-administer a dose of a PRN medical substance. The function of the patient control interface 30 is explained in more detail in FIG. 2.

FIG. 2 depicts an example patient control interface 30 according to certain aspects of the present disclosure. In this example, the patient control interface is a wireless handheld 30 that may be similar in size to a television remote control. This example handheld 30 is configured to enable the patient 10 of FIG. 1 to administer a dose of either of two PRN medications wherein the ventilator 15 of FIG. 1 and an aerosol generator (not shown in FIG. 1) have been configured according to a doctor's prescription. The handheld 30 is configured such that the names of the two PRN medical substances are shown in displays 32A and 32B. There are 3 buttons on the example handheld 30. The “1” button 36A initiates the administration of the medical substance shown in display 32A and the “2” button 36B initiates the administration of the medical substance shown in display 32B. A label 38 is provided, in this example, to further instruct the patient as to the function of the button 36A and 36B. These buttons may be illuminated and/or color-coded to assist the patient 10 in understanding their function or operating them at night or in reduced illumination. For instance, the nurse call button 52 may be red to indicate that it is the button to push if the situation is urgent or the patient is in distress.

In the example of FIG. 2, the ventilator 15 has been configured with lock-out time periods for each of the medications identified in displays 32A and 32B. Displays 34A and 34B display the amount of time remaining before another dose of the medications shown in displays 34A and 34B, respectively, can be administered. Pressing buttons 36A and 36B while time remains in the respective lock-out time period will not initiate the administration of a dose of the respective medical substance.

As using a ventilator 15 may be inherently uncomfortable, it may be desirable to provide assurance to the patient 10 that they are not at risk of injury and so have less incentive to self-administer a PRN medical substance. To this end, feedback is provided by displaying health parameters of the patient 10 that are, in this example, the measured value of the patient's blood oxygen level 40 and the measured value of the patient's breathing rate 42. To provide an intuitive guide to the desired ranges of these health parameters, the displays 40 and 42 have, in this example, adjacent colored bars that are red to indicate undesirable ranges and green to indicate desirable ranges of each parameter. In this example, blood oxygen 40 has a red bar 44 and a green bar 46 while breathing rate has two red bars 48, as the patient's breathing rate could be undesirably high or low, as well as a green target bar 50. By examining the displays 40 and 42, the patient 10 and their family can verify that the patient 10 is not in physical danger although they may be in discomfort.

FIG. 3 is a block diagram of a ventilator 15 and an aerosol generator 260 configured to be controlled by a patient 10 according to certain aspects of the present disclosure. The patient 10 is wearing a patient device 16, such as depicted in FIG. 1, that may be any of the masks or intubation devices known to those of ordinary skill in the art for introducing gas into the lungs of a patient, including full-face or partial-face masks, an endotracheal tube, or a tracheotomy tube. The ventilator 15 is shown in this example as ventilator 15, comprising a gas control module 215, a processor 205 and memory 210, a clinician interface 220, and a communication module 235. The gas control module 215, in this example, selectively delivers gas at a selected pressure, humidity, temperature, and flow rate to the patient device 16 through hose 230. The connection between ventilator 15 and patient device 16 is, in this example, accomplished by a hose 230 from the gas control module 215 to the patient device 16. In certain embodiments, hose 230 includes an inspiratory hose and an expiratory hose (not shown separately) such that the patient's exhaled gas is returned to the ventilator 15. The gas may be ambient air, air enhanced with a specified amount of oxygen, or a mixture of one or more of air, oxygen, and other gases such as nitrogen or helium.

In certain embodiments, some of the elements of ventilator 15 will be omitted while in certain other embodiments, additional elements are incorporated into ventilator 15. In certain embodiments, elements such as the clinician interface 220 may be external to the ventilator 15. In certain embodiments, elements such as the clinician interface 220 may be provided by another piece of equipment such as a standard desktop computer or a handheld device such as a cellular phone. In certain embodiments, the elements shown may be combined or functions from one element may be accomplished by another element. The elements 205, 210, 215, 220, and 235 are shown as interconnected by a bus 255, enabling each element to talk to any other element on the bus. In certain embodiments, some or all of the elements 205, 210, 215, 220, and 235 may be interconnected only with one or more of the other elements by any methods of communication known to those of ordinary skill in the art, including multiple parallel buses and serial data links.

Ventilator 15 is also coupled, in this example, from communication module 235 to a patient control interface 30 through a communication link 245. In certain embodiments, such as the wireless handheld 30 of FIGS. 1 and 2, communication link 245 may be an optical or radio-frequency one-way or bidirectional link. In certain other embodiments, the patient control interface 30 is a part of the ventilator 15. In certain other embodiments, the patient control interface 30 is an alternate screen display on the clinician interface 220. In certain other embodiments, the patient control interface 30 is a display on a separate computer.

An aerosol generator 260 is coupled to the patient device 16 through a hose 270 that, in this example, connects to the inspiratory hose of air hose 230 at a point between the ventilator 15 and the patient device 16. In certain other embodiments, this connection is at the patient device 16. In certain other embodiments, the aerosol generator 260 is a part of the ventilator 15 and hose 270 connects to the gas control module 215. Aerosol generator 260 is, in this embodiment, coupled to a reservoir 265 that is configured to store and handle the medical substance. In the example of FIG. 3, the medical substance is a liquid. In certain other embodiments, the medical substance may in the form of a solid, a powder, a gas, a solid medication suspended in a liquid, or a combination of these forms of matter.

In this example, the patient control interface 30 is coupled to the aerosol generator through communication link 275. When manipulated by the patient 10, the patient control interface 30 controls the aerosol generator 260 to administer the medical substance that is contained in reservoir 265, whereupon the aerosol generator 260 creates an aerosol that comprises the medical substance for reservoir 265 and delivers this aerosol through hose 270 to the patient 10. In certain embodiments, the aerosol generator 260 is connected (not shown) to the processor 205 and controlled by the processor 205 of the ventilator 15, wherein the patient control interface 30 sends a command to the processor 205 through communications module 235 and the processor 205 controls the aerosol generator 260 to administer the medical substance that is contained in reservoir 265. In certain other embodiments, the operation of the gas control module 215 is adjusted in concert with the operation of the aerosol generator 260 to enhance the delivery of the medical substance. These adjustments may include variations in the operation of the gas control module 215 such as changes in the flow rate and pressure of the gas supplied by the gas control module 215, regulation of the relative pressure of the gas supplied by the gas control module 215 with respect to the pressure of the aerosol delivered by the aerosol generator 260, and cessation of humidification of the supplied gas by the gas control module 215 while the aerosol generator 260 is delivering aerosol.

In certain embodiments, the communication module 235 of ventilator 15 is linked to an external server or database 250 through a network 250 such as an Ethernet wired or wireless network 253. In certain other embodiments, the processor 205 retrieves executable instructions, information on prescribed operating parameters for a specific patient 10, or other data or information related to the operation of ventilator 15 or to the patient 10. In certain other embodiments, the processor 205 transmits information to the database 250, such as a history of operation, time and dose of each administration of the medical substance, a log of patient actions, or a record of actuations of the patient control interface 30 regardless of whether the medical substance was administered.

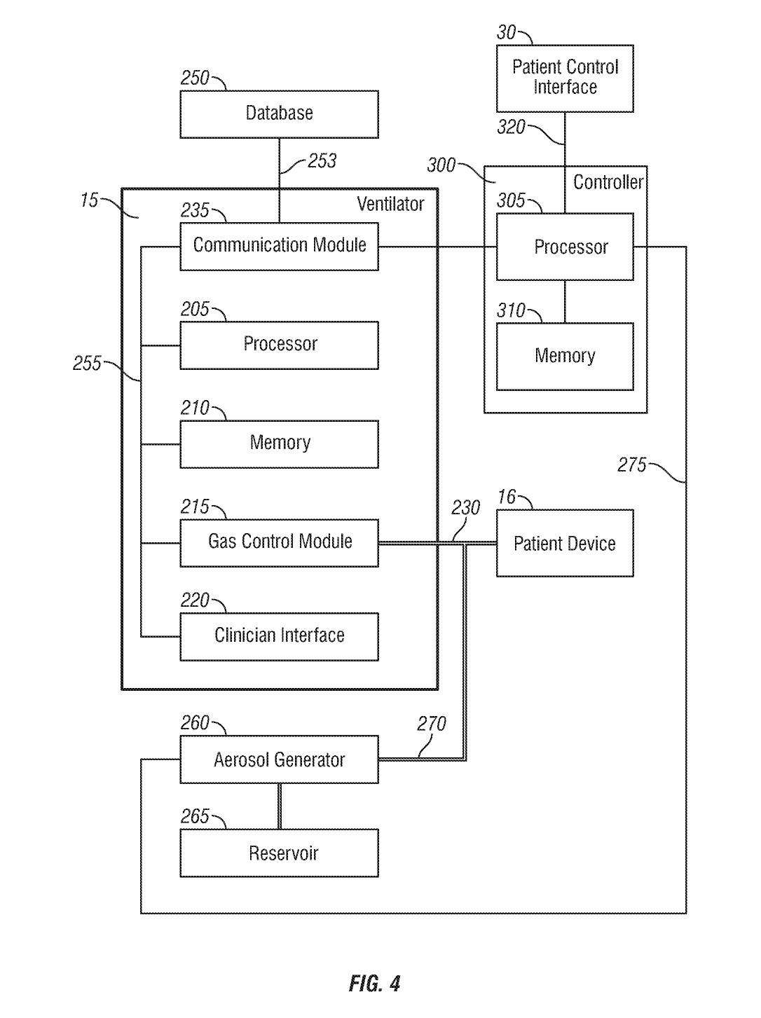

FIG. 4 is a block diagram of a ventilator controller 300 configured to control a ventilator 15 and an aerosol generator 260 according to certain aspects of the present disclosure. Whereas in FIG. 3, the patient control interface 30 communicated directly with one or both of the ventilator 15 and the aerosol generator 260, the system depicted in FIG. 4 includes a ventilator controller 300. The patient control interface 30 sends a signal over a wired or wireless communication link 320 to the processor 305, which also is coupled to memory 310 wherein are stored the instructions for controlling the ventilator 15 and aerosol generator 260. The processor 305 of ventilator controller 300 communicates with the processor 205 through a wired or wireless link 315 and communication module 235. The processor 305 also communicates with the aerosol generator 260 over a wired or wireless communication link 275. In response to a signal from the patient control interface 30 and in accordance with the instructions retrieved from memory 310, the processor 305 sends a signal to processor 205 to configure the ventilator 15 and sends a signal to the aerosol generator 260 to aerosolize and dispense a dose of a medical substance. In certain embodiments, the operation of the ventilator 15 is not changed during administration of an aerosolized medical substance and therefore no signal is sent from the processor 305 to the processor 205. In certain other embodiments, the ventilator controller 300 is not coupled to the ventilator 15. In certain embodiments, the link 315 comprises the network 253 and the ventilator controller 300 communicates with ventilator 15 through link 253. In certain embodiments, the ventilator controller 300 is connected to network 253. In certain other embodiments, the ventilator controller 300 is integrated with the aerosol generator 260.

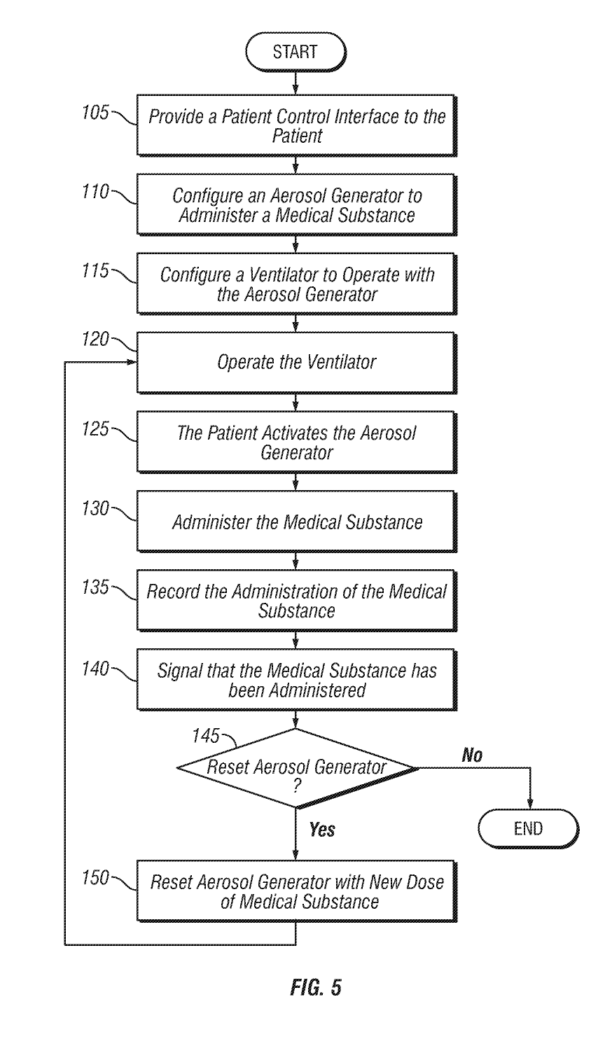

FIG. 5 is a flow chart of the methodology of a patient 10 controlling the administration of a medical substance according to certain aspects of the present disclosure. This process is described in relation to the ventilator 15 and aerosol generator 260 shown in FIG. 3. In step 105, a nurse or other caregiver provides a patient with a patient control interface 30. In step 110, the nurse configures an aerosol generator 260 to administer a dose of the medical substance and set any lock-out time intervals or limits associated with administration of the medical substance. In step 115, the nurse configures ventilator 15 to operate with aerosol generator 260. In certain embodiments, processor 215 controls both the aerosol generator 260 and the ventilator 15. In certain embodiments, the aerosol generator 260 operates independently of the ventilator 15. In certain embodiments, the aerosol generator 260 is connected to ventilator 15 such that information is exchanged between the aerosol generator 260 and the ventilator 15, the information comprising one or more of monitored health parameters of the patient 10, administration instructions related to the medical substance, and operational settings. In certain other embodiments, the nurse configures the aerosol generator 260 such that it will administer a dose of medical substance only if one or more monitored health parameters of the patient 10 are within specified limits.

After the ventilator 15 and aerosol generator 260 are configured in steps 110 and 115, the ventilator 15 is operated in step 120. In certain embodiments, the ventilator 15 is operating prior to the start of this process and step 120 comprises switching the mode of operation to enable the administration of the medical substance by the aerosol generator 260. The ventilator 15 continues to operate in step 120 until, in step 125, the patient 10 activates the aerosol generator 260 through the patient control interface 30, whereupon the aerosol generator 260 administers the medical substance if the specified lock-out time period has elapsed. After completing the administration of the medical substance in step 130, the aerosol generator 260 records the administration in step 135 and, in step 140, provides a signal that the medical substance has been administered. In certain embodiments, the aerosol generator 260 transmits information about the administration of the medical substance through the ventilator 15 to the database 250. In certain embodiments, the aerosol generator 260 activates a visual indicator on the aerosol generator 260 or the ventilator 15. In certain embodiments, the aerosol generator 260 sends a message to the nurse via a pager message, an Email message, or other form of communication known to those of ordinary skill in the art. The nurse, in step 145, decides whether to reset the aerosol generator, following the “YES” path to step 150, or to terminate the patient-controlled administration of the medical substance, whereupon the process follows the “NO” path to the end. In step 150, the nurse loads another dose of the medical substance into the reservoir 265 and resets the aerosol generator 260. In certain embodiments, the reservoir 265 holds more than one dose and the aerosol generator does not need to be reset between doses, in which case the process moves directly from step 135 to step 120 (this path not shown).

It is understood that the specific order or hierarchy of steps in the processes disclosed is an illustration of exemplary approaches. Based upon design preferences, it is understood that the specific order or hierarchy of steps in the processes may be rearranged and some of the steps may be performed simultaneously or omitted without departing from the scope of the claimed invention. The accompanying method claims present elements of the various steps in a sample order, and are not meant to be limited to the specific order or hierarchy presented.

The previous description is provided to enable any person skilled in the art to practice the various aspects described herein. While the foregoing has described what are considered to be the best mode and/or other examples, it is understood that various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the terms “a set” and “some” refer to one or more. Pronouns in the masculine (e.g., his) include the feminine and neuter gender (e.g., her and its) and vice versa. Headings and subheadings, if any, are used for convenience only and do not limit the invention.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

A phrase such as an “aspect” does not imply that such aspect is essential to the subject technology or that such aspect applies to all configurations of the subject technology. A disclosure relating to an aspect may apply to all configurations, or one or more configurations. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. A phrase such an embodiment may refer to one or more embodiments and vice versa.

The word “exemplary” is used herein to mean “serving as an example or illustration.” Any aspect or design described herein as “exemplary” is not necessarily to be construed as preferred or advantageous over other aspects or designs.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A method of controlling the administration of a medical substance, the method comprising the steps of:
configuring an aerosol generator to aerosolize a medical substance and administer the aerosolized medical substance to a patient using a ventilator;
providing the patient with a patient control interface through which a patient initiates the administration of a dose of the aerosolized medical substance; and
configuring a processor to control the ventilator and the aerosol generator in response to the patient control interface such that the patient controls the administration of the aerosolized medical substance in accordance with limits on the administration of the medical substance.

2. The method of claim 1, wherein the step of configuring the processor further comprises specifying a lock-out time period such that a dose of the aerosolized medical substance is administered only after the lock-out time period has elapsed since the last prior administration of the aerosolized medical substance.

3. The method of claim 2, further comprising the step of displaying the amount of time remaining in the lock-out period.

4. The method of claim 1, wherein the medical substance is a medication.

5. The method of claim 1, further comprising the step of measuring at least one monitored parameter that is associated with the health of the patient, and wherein the step of configuring the processor further comprises specifying at least one limit for the at least one monitored parameter such that a dose of the aerosolized medical substance is administered only when the at least one monitored parameter is within the at least one limit.

6. The method of claim 1, wherein the step of configuring the processor further comprises selecting a dosage range such that the patient uses the patient control interface to vary the dose of the aerosolized medical substance to be administered within the dose range.

7. The method of claim 1, further comprising the steps of:
measuring at least one health parameter that is associated with the current health of the patient; and
displaying the at least one health parameter.

8. A ventilation system for use by a patient, comprising:
a patient device attached to the patient, the patient device configured to introduce gas into the lungs of the patient;
a gas control module fluidically coupled to the patient device, the gas control module configured to controllably provide a gas to the patient device according to at least one operating parameter;
an aerosol generator coupled to the patient device, the aerosol generator configured to administer a dose of an aerosolized medical substance;
a patient control interface configured to control the at least one operating parameter of the gas control module and to be accessible by the patient; and
a processor coupled to the gas control module, the aerosol generator, and the patient control interface, the processor configured to operate the gas control module and the aerosol generator in response to the patient control interface.

9. The ventilation system of claim 8, wherein:
the gas control module is configured to measuring a health parameter that is associated with the health of the patient;
the patient control interface is further configured to display the health parameter.

10. The ventilation system of claim 8, further comprising a memory coupled to the processor, the memory configured to store one or more executable instructions, wherein the processor is further configured to retrieve the instructions from the memory and operate the gas control module and the aerosol generator in accordance with the retrieved instructions.

11. The ventilation system of claim 8, wherein:
the gas control module is configured to measuring a health parameter that is associated with the health of the patient;
the aerosol generator is further configured to administer the aerosolized medical substance only if the measured health parameter is within present limits.

12. The ventilation system of claim 8, wherein the aerosol generator is further configured to administer a dose of the aerosolized medical substance only after a specified lock-out time period has elapsed since the last prior administration of the aerosolized medical substance.

13. A computer-readable medium having computer-executable instructions stored thereon for execution by a processor to perform a method of controlling the administration of a medical substance, the method comprising the steps of:
configuring an aerosol generator to aerosolize a medical substance and administer the aerosolized medical substance to a patient using a ventilator;
providing the patient with a patient control interface through which a patient initiates the administration of a dose of the aerosolized medical substance; and
configuring a processor to control the ventilator and the aerosol generator in response to the patient control interface such that the patient controls the administration of the aerosolized medical substance in accordance with limits on the administration of the medical substance.

14. The computer-readable medium of claim 13, wherein the step of configuring the processor further comprises specifying a lock-out time period such that a dose of the aerosolized medical substance is administered only after the lock-out time period has elapsed since the last prior administration of the aerosolized medical substance.

15. The computer-readable medium of claim 14, further comprising the step of displaying the amount of time remaining in the lock-out period.

16. The computer-readable medium of claim 13, wherein the medical substance is a medication.

17. The computer-readable medium of claim 13, further comprising the step of measuring at least one monitored parameter that is associated with the health of the patient, and wherein the step of configuring the processor further comprises specifying at least one limit for the at least one monitored parameter such that a dose of the aerosolized medical substance is administered only when the at least one monitored parameter is within the at least one limit.

18. The method of claim 13, wherein the step of configuring the processor further comprises selecting a dosage range such that the patient uses the patient control interface to vary the dose of the aerosolized medical substance to be administered within the dose range.

19. The method of claim 13 further comprising the steps of:
measuring at least one health parameter that is associated with the current health of the patient; and
displaying the at least one health parameter.

20. A method of controlling the administration of a medical substance to a patient using a ventilator, the method comprising the steps of:
configuring an aerosol generator to aerosolize a medical substance and administer the aerosolized medical substance to the patient;
providing the patient with a patient control interface through which a patient initiates the administration of a dose of the aerosolized medical substance; and
configuring a processor to control the aerosol generator in response to the patient control interface such that the patient controls the administration of the aerosolized medical substance in accordance with limits on the administration of the medical substance.

21. A ventilator controller configured to control a ventilator, comprising:
an aerosol generator configured to couple to a patient device that is configured to introduce gas into the lungs of the patient, wherein the patient device is also coupled to a ventilator, the aerosol generator further configured to administer a dose of an aerosolized medical substance;
a memory configured to store one or more executable instructions and data;
a patient control interface configured to initiate administration of a dose of an aerosolized medical substance and to be accessible by the patient; and
a processor coupled to the aerosol generator, the memory, and the patient control interface, the processor configured to retrieve the instructions and data from the memory and operate the aerosol generator in response to the patient control interface.

22. The ventilator controller of claim 21, wherein the processor is further configured to be coupled to the ventilator and to modify the operation of the ventilator when a dose of the aerosolized medical substance is being administered.

23. The ventilator controller of claim 21, wherein the processor is further configured to administer a dose of the aerosolized medical substance only after a specified lock-out time period has elapsed since the last prior administration of the aerosolized medical substance.
